(19) United States
(12) Patent Application Publication
(10) Pub. No.: US 2008/0274148 A1
(43) Pub. Date: Nov. 6, 2008

(54) Microcapsule Composition

(76) Inventor: Jonathan Edward Creeth, Surrey (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/915,011

(22) PCT Filed: May 19, 2006
(86) PCT No.: PCT/EP2006/005048

§ 371 Date: Nov. 20, 2007

(30) Foreign Application Priority Data
May 23, 2005 (GB) 0510498.9

(51) Int. Cl. A61K 8/11; A61Q 11/00

(52) U.S. Cl. 424/401; 424/49

(57) ABSTRACT

The present invention comprises an oral care composition comprising a microcapsule containing a coated water soluble dye particle that is incorporated into a liquid hydrophobic material. Suitably the microcapsule containing the water soluble dye forms part of a clear gel toothpaste formulation.

The present invention relates to a microcapsule system and the use of microcapsules in oral care compositions. Specifically the present invention relates to a microcapsule system comprising a treated water soluble dye (hydrophilic) particle incorporated into or surrounded by a hydrophobic liquid carrier medium. This microcapsule is then suspended within an aqueous clear gel composition. Said composition shows excellent microcapsule colour integrity with minimal colour migration out of the capsule into the clear gel composition.

Microcapsules are desirable for oral care compositions for many reasons. They are particularly useful as delivery devices for delivering materials that may provide cosmetic, sensory or protective benefits to the oral cavity, for example flavouring materials and essential oils. When the microcapsules contains a flavour they may contain a hydrophobic or oily material in which to solubilise the flavouring material.

Microcapsules can also enhance the visual appeal of an oral care composition. As oral care compositions generally contain high amounts of water or aqueous phases, when water soluble dyes are used in microcapsules in such compositions it is necessary to physically isolate the dye in order to prevent it from migrating and mixing with the water in the surrounding phases. If the dye is not isolated it can result in bleeding or staining of the whole or a large part of the composition. This problem is particularly apparent if the dye-containing capsule is to be part of a clear gel composition. Such compositions have become increasingly popular in recent years because they allow manufacturers to impart increased distinctiveness to their product.

It is not however possible to prepare hydrophobic microcapsules containing oil in the liquid state with a water soluble dye for a clear gel composition as hydrophilic materials cannot be incorporated into hydrophobic materials as they will not mix.

The present invention therefore seeks to prepare a clear gel composition containing a hydrophobic capsule containing liquid oil and further containing a water soluble dye.

Capsule systems have been proposed in the prior art such as WO02/060573 that discloses a capsule-in-capsule system consisting of an external capsule envelope and internal capsules located therein. The internal capsules are located in a medium which can be a solid or liquid medium and have a capsule envelope which completely encloses an active ingredient such as a fragrance, essential oil, colouring or antibacterial agent.

EP-A-1020177 discloses a multi-layered soft capsule that is effective in eliminating bad breath. The multi-layered soft capsule comprises a first soft capsule layer that is soluble in the oral cavity and a second soft capsule layer that is soluble in the stomach. The contents in both soft capsule are completely encapsulated by the soft layers and are both effective in eliminating bad breath.

We have since found that it is not necessary, as shown in the prior art, to completely surround or protect the ingredient, for example the water soluble dye, that is located within the microcapsule. It has been shown that it is only necessary to partially coat the ingredient to give the desired effect of non-leaching, bleeding or migration. This will create visually appealing microcapsules of this type described below.

It is an object of the present invention to provide a coloured microcapsule that is both visually and cosmetically acceptable in a clear gel toothpaste formulation. The microcapsule will provide a composition that gives excellent colour intensity without any of the problems mentioned above that are encountered when a water soluble dye is used in an oral care composition, particularly a clear gel composition. It also provides an excellent system for delivery of active substances.

Accordingly the present invention comprises an oral care composition comprising a microcapsule containing a coated water soluble dye particle that is incorporated into a liquid hydrophobic material.

The external wall of the microcapsule can be made of any material providing the following characteristics. It must be compatible with the ingredients typically found in toothpaste formulations, it must be strong enough to withstand commercial toothpaste manufacturing and filling processes yet soft enough to burst on brushing and it must be storage stable. It must also be sufficiently transparent to allow the colour of the water soluble dye to show through. Suitably the external capsule wall is made from gum, agar type materials, alginates or from a gelatin-gum arabic coacervate. Gelatin-gum arabic coacervate is the preferred material.

The microcapsules are preferably present in an amount of from 0.1 to 10% w/w of the total composition.

The dye particle is treated prior to being surrounded in the hydrophobic material. The term treated can include various ways of protecting and coating the particle and making it impervious to water. A preferred treatment is by forming a matrix particle of the dye with a substrate and spray congealing the mixture. This will form a fine particle of very fine particles of the dye in a matrix of the substrate. The spray congealing process is an easy, efficient and quick process. U.S. Pat. No. 6,037,000 discloses such a process of treating a dye particle by spray congealing and the contents of that patent are by reference incorporated into this application. However it is fully appreciated in U.S. Pat. No. 6,037,000 that “the spray congealing process is not 100% effective”, that is, it does not completely coat the particle. Spray congealing will not completely screen the dye particle from the surrounding medium. Small areas or gaps of untreated particle will inevitably remain exposed. This surprisingly does not effect colour migration of the dye within the spray congealed particle once the particle is incorporated into a microcapsule containing a hydrophobic liquid material.

The hydrophobic material in which the coated dye particle is incorporated into is an oil or wax or mixture thereof. Preferably the hydrophobic material is an oil although it may be a wax provided it is liquid during manufacture of the microcapsules. Such oils include vegetable oils, mineral oils or silicone oils. Edible vegetable oils are preferred, and include palm oil, sunflower oil, safflower oil, sesame oil, rapeseed oil, grape-seed oil, shea butter, flavour oils such as mint, clove, aniseed, and mixtures thereof. Sunflower oil is preferred because it forms a clean emulsion in water and will not leave any remaining residues. Suitable food grade sunflower oil is commercially available

Suitably the oil is present in the capsules in an amount ranging from 70-99% w/w, preferably in the range 80-98% w/w, more preferably from 85-95% w/w.

The diameter of the microcapsule is about 1 m to about 10 mm, preferably from 10 m to 5 mm, more preferably from 150 m to 2.5 mm.

There are many water soluble dyes that may be used in oral care compositions. These will be well known to those skilled in the art. The present invention is relevant to all water soluble dyes. Preferred dyes are FD&C Blue No. 1, Blue No. 2, Green No. 3, Green No. 6, Red No. 3, Red No. 10, Yellow No. 5, Yellow No. 6, Yellow No. 7, Yellow No. 8 and Yellow No. 10. A preferred dye is FD&C Blue No. 1.

In addition to the microcapsule the dentifrice may also contain other additives or excipients conventional in the field of oral care products such as a fluoride source. For a composition for use in cleaning natural teeth the inclusion of a fluoride ion source is also desirable because of its caries protection activity, but for a composition for use in cleaning artificial dentures such activity is not necessary. The fluoride ion source may be provided by an alkali metal fluoride, preferably sodium fluoride, an alkali metal monofluorophosphate, stannous fluoride, amine fluoride and the like. Preferably, however, the fluoride ion source is an alkali metal fluoride, most preferably sodium fluoride. The fluoride ion source serves in a known manner for caries protection. Preferably, the fluoride ion source will be used in an amount to provide an effective amount such as an amount sufficient to provide from about 25 ppm to about 3500 ppm, preferably about 1100 ppm, as fluoride ion. For example the formulation may contain 0.1-0.5 wt % of an alkali metal fluoride such as sodium fluoride.

Preferably the pH of the composition is from 4 to 10.5, more preferably from 5.5 to 9.5. Typically the composition may contain up to 0.5 wt. % of sodium hydroxide to provide a suitable pH.

In compositions of the present invention i.e. those which can be extruded onto a toothbrush, the orally acceptable vehicle may be of a generally conventional composition e.g. comprising a thickening agent, a binding agent and a humectant. Preferred binding agents include for example natural and synthetic gums such as xanthan gums, carageenans, alginates, cellulose ethers and esters. Preferred humectants include glycerin, sorbitol, propylene glycol and polyethylene glycol. A preferred humectant system consists of glycerin, sorbitol and polyethylene glycol. If the composition is a clear gel the amount and type of humectant should be carefully chosen to match the refractive index of the abrasive particles and any thickening particles contained within the formulation.

In addition, the orally acceptable vehicle may optionally comprise one or more surfactant, sweetening agent, flavouring agent, anticaries agent (in addition to the fluoride ion source), anti-plaque agent, anti-bacterial agent such as triclosan or cetyl pyridinium chloride, tooth desensitizing agent, colouring agents and pigment. Useful surfactants include the water-soluble salts of alkyl sulphates having from 10 to 18 carbon atoms in the alkyl moiety, such as sodium lauryl sulphate, but other anionic surfactants as well as non-ionic, zwitterionic, cationic and amphoteric surfactants may also be used.

It is preferred to use a thickening silica as the thickening agent. So called “thickening silicas” are known silicas which have relatively little abrasive effect compared with known abrasive silicas such as Zeodent 113™, but provide a thickening effect on the composition. Suitable thickening silicas are known and include those marketed by Huber under the tradename Zeodent, e.g. Zeodent 167, Zeofree 153B and by Degussa AG under the trade name SIDENT®, e.g. SIDENT 22S®, and by Grace-Davison Chemical Division under the trade name SYLOBLANC®, e.g. SYLOBLANC 15®, respectively. For example the composition may contain up to ca. 20 wt % of a thickening silica, typically 2-15 wt %)

The composition of the invention may contain an abrasive material, for example known types of “abrasive silica” commonly used in toothpaste compositions e.g. Zeodent 113™ as mentioned above.

The additives or excipients used in any given composition will be compatible both with each other and with the essential ingredients of the composition such that there is no interaction which would impair the performance of the active ingredients. All additives or excipients must of course be of low toxicity and of sufficient purity to render them suitable for human use.

The preparation of the microcapsules may be carried out in accordance with well-known encapsulating processes, preferably by coacervation. Such processes are widely known and described in many publications, for example by the National Cash Register Co., in their brochure “NCR Mic roencapsulation Applications” (1966).

The oral care composition of the invention is a gel. The composition is a clear gel that comprises hydrophobic microcapsules. The microcapsules will contain at least one dye particle that has been coated by a spray-congealing process. Preferably the hydrophobic microcapsule will each contain 0.01-20% dye particles, all individually spray-congealed. More preferably the hydrophobic microcapsule will each contain 0.1-10% dye particles, most preferably 0.5-6%. The final composition may be presented in any form that is suitable for application to the oral cavity. Suitably the composition is in the form of a conventional toothpaste-type composition that can be squeezed from a collapsible tube.

The clear gel compositions of the present invention are prepared first by admixing according to conventional practice the ingredients of the composition in a suitable vessel such as a stainless steel tank provided with a mixer. The coloured hydrophobic microcapsules are then mixed with the clear gel composition and suitably packaged and stored as required. This will produce a storage-stable semi-solid extrudable material useful as a dentifrice.

The present invention is illustrated by the following examples but is not limited thereby.

### EXAMPLES

1. Clear Gel Formulation containing capsules

| Ingredient Name | % w/w |
|---|---|
| Sorbitol Solution 70% Non Crystallising | 63.16 |
| Abrasive silica | 14.00 |
| Water | 7.20 |
| Thickening silica | 5.50 |
| Polyethylene Glycol 300 | 3.00 |
| Sodium Lauryl Sulphate | 1.50 |
| Flavour | 1.00 |
| Xanthan Gum | 0.60 |
| Disodium Phosphate Anhydrous | 0.60 |
| Sodium Saccharin | 0.20 |
| Sodium Fluoride | 0.24 |
| Microcapsules (0.3% blue lake, spray | 3.00 |
| congealed) | |
| Total | 100.00 |

The microcapsules containing spray-congealed FD&C Blue No. 1 dye particles incorporated into sunflower oil are carefully added by mixing to the clear gel composition as the final step.

2. Clear Gel Formulation containing capsules

| Ingredient Name | % w/w |
|---|---|
| Sorbitol Solution 70% Non Crystallising | 63.16 |
| Abrasive silica | 14.00 |
| Water | 7.20 |
| Thickening silica | 5.50 |
| Polyethylene Glycol 300 | 3.00 |
| Sodium Lauryl Sulphate | 1.50 |
| Flavour | 1.00 |
| Xanthan Gum | 0.60 |
| Disodium Phosphate Anhydrous | 0.60 |
| Sodium Saccharin | 0.20 |
| Sodium Fluoride | 0.24 |
| Microcapsules (0.2% blue lake, non-spray | 3.00 |
| congealed) | |
| Total | 100.00 |

The microcapsules containing spray-congealed FD&C Blue No. 1 dye particles incorporated into sunflower oil are carefully added by mixing to the clear gel composition as the final step.

Dye particles that are not spray-congealed prior to incorporation into the hydrophobic microcapsule show dye migration and hence clearly visible colour leaching into the clear gel composition within days of manufacture.

1. An oral care composition comprising a microcapsule containing a coated water soluble dye particle that is incorporated into a hydrophobic liquid material.

2. An oral care composition as claimed in claim 1, wherein the water soluble dye particle is coated using a spray congealed process.

3. An oral care composition as claimed in claim 2, wherein the hydrophobic material is an oil.

4. An oral care composition as claimed in any one of the above claims, wherein the hydrophobic oil is an edible vegetable oil and includes palm oil, sunflower oil, safflower oil, sesame oil, rapeseed oil, grape-seed oil, shea butter, flavour oils such as mint, clove or aniseed and mixtures thereof.

5. An oral care composition as claimed in claim 4, wherein the hydrophobic oil is present in the capsules in an amount ranging from 70-99% w/w.

6. An oral care composition as claimed in claim 5, wherein the hydrophobic oil is present in the capsules in an amount ranging from 80-98% w/w.

7. An oral care composition as claimed in any one of the above claims wherein the microcapsules are present in an amount of from 0.1 to 10% w/w of the total composition.

8. An oral care composition as claimed in claim 7, which is a clear gel composition comprising a microcapsule containing at least one coated dye particle.
